(19) United States
(12) Patent Application Publication
(10) Pub. No.: US 2015/0306260 A1
(43) Pub. Date: Oct. 29, 2015

(54) METHOD FOR STERILIZING MEDICAL DEVICE MADE OF ESTER RESIN

(71) Applicant: NIPRO CORPORATION, Osaka-shi, Osaka (JP)

(72) Inventors: Yoshihiko Sano, Osaka (JP); Toshiaki Masuda, Shiga (JP); Takeshi Yamaguchi, Osaka (JP)

(73) Assignee: NIPRO CORPORATION, Osaka-shi, Osaka (JP)

(21) Appl. No.: 14/650,247

(22) PCT Filed: Dec. 5, 2013
(86) PCT No.: PCT/JP2013/007160

§ 371 Date: Jun. 5, 2015

(30) Foreign Application Priority Data
Dec. 7, 2012 (JP) 2012-268148

(51) Int. Cl. A61L 2/08

(52) U.S. Cl. CPC A61L 2/081

(57) ABSTRACT

A medical device made of an ester resin, such as a hollow-fiber-type blood treatment device, is put into a packaging material made of a gas-impermeable material, and, with at least a reducing gas, such as hydrogen gas, being further enclosed therein, the packaging material is hermetically sealed to provide a medical device package. The medical device package is exposed to radiation to sterilize the inside thereof. In the medical device package, an oxygen scavenger may be further enclosed. The reducing gas may be mixed with an inert gas and enclosed as a mixed gas. As a result, it becomes possible to effectively suppress the generation of by-products, such as acetic acid, from the ester resin.

### TECHNICAL FIELD

The present invention relates to a method for sterilizing a medical device produced from an ester resin (medical device made of an ester resin) by exposure to radiation.

### BACKGROUND ART

Ester resins having an ester bond in the molecule have been conventionally used for various medical devices. Ester resins have various physical properties that are suitable for use in medical devices. In addition, as compared to glass, etc., they have excellent formability and workability, are light in weight, and are relatively inexpensive.

Taking a blood treatment device as an example of a medical device, in the blood purification therapy in the treatment of renal insufficiency, etc., for the purpose of removing uremic toxin or waste from the blood, modules (blood treatment devices) including a dialysis membrane or an ultrafiltration membrane as a separating material, such as hemodialyzers, hemofilters, and hemodiafilters, are widely used. For the dialysis membrane or the ultrafiltration membrane of such a module, cellulose-based natural materials or various synthetic polymers are used. In particular, modules including a hollow-fiber-type membrane as a separating material (hollow-fiber-type blood treatment devices) have advantages in terms of the reduction of the amount of extracorporeally circulating blood, the high efficiency of the removal of substances from the blood, and further the productivity in module production, for example. Therefore, they are highly important in the field of dialyzers.

Hollow-fiber-type blood treatment devices have to be completely sterilized before use, and thus various sterilization methods are used. Among them, a method of sterilization by exposure to radiation can treat a hollow-fiber-type blood treatment device in a packaged state and also have high sterilization effects, and thus has been employed as one of the preferred sterilization methods. However, in this sterilization method, due to exposure to radiation, some of the members forming the hollow-fiber-type blood treatment device may be degraded, or generate by-products. Thus, techniques for sterilization by exposure to radiation, which are intended to suppress the degradation of a hollow-fiber-type blood treatment device or the generation of by-products, etc., have been known.

For example, Patent Literature 1 proposes, in order to suppress the generation of harmful by-products while maintaining sterilization efficiency by exposure to radiation, a sterilization method including the steps of irradiating a package while maintaining an atmosphere having a reduced oxygen concentration in the package; and, after irradiation, further reducing the oxygen concentration with an oxygen scavenger while maintaining the sterilized state of the package.

In addition, Patent Literature 2 proposes, in a semipermeable membrane containing a hydrophobic polymer and a hydrophilic polymer, in order to suppress the decomposition of the polymers and suppress the elution of the hydrophilic polymer, a sterilization method in which the semipermeable membrane is hydrated with water in an amount of 100 to 600% of the membrane's own weight, and an inert gas atmosphere is made in a dialyzer, followed by gamma irradiation.

### CITATION LIST

### Patent Literature

PTL 1: International Publication No. WO 98/058842 pamphlet

PTL 2: Japanese Laid-Open Patent Application Publication No. 2001-170167

### SUMMARY OF INVENTION

### Technical Problems

Here, as hollow fibers for a hollow-fiber-type blood treatment device, those made of acetyl cellulose, which is a derivative of cellulose, are known. Acetyl cellulose is an ester resin in which acetic acid is linked to a hydroxyl group (—OH) in the cellulose molecule by an ester bond. When hollow fibers made of acetyl cellulose are exposed to radiation, part of acetyl cellulose is decomposed to generate acetic acid. Although it is desired that the transmitted liquid is as close to neutral as possible, as a result of the generation of acetic acid, the pH of the transmitted liquid that has passed through the hollow fibers may shift to the acidic side (the pH may decrease). In addition, the decomposition of acetyl cellulose due to exposure to radiation also causes the degradation of hollow fibers.

According to the sterilization method of Patent Literature 1 mentioned above, the degradation of acetyl cellulose and the generation of acetic acid due to exposure to radiation may not be effectively suppressed. In addition, according to the sterilization method of Patent Literature 2 mentioned above, because hollow fibers have to be moisturized with a large amount of water, the sterilizing process is complicated. Further, in Patent Literature 2, only carboxymethyl cellulose is mentioned as an example of a hydrophilic polymer that is a cellulose derivative, and polyvinyl pyrrolidone is mentioned as a preferred polymer. Therefore, it is not clear whether the generation of acetic acid from acetyl cellulose can be effectively suppressed by this sterilization method.

The present invention has been accomplished to solve such problems, and an object thereof is to propose, in the case where a medical device made of an ester resin, such as a hollow-fiber-type blood treatment device including hollow fibers made of acetyl cellulose, when the medical device is sterilized by exposure to radiation, a sterilization method is capable of effectively suppressing the degradation of the ester resin and also the generation of by-products, such as acetic acid (carboxylic acid), due to the decomposition of the ester resin.

### Solution to Problems

In order to solve the problems, the method for sterilizing a medical device made of an ester resin according to the present invention is configured as follows: a method for sterilizing a medical device made of an ester resin, including: hermetically sealing a medical device made of an ester resin in a packaging material made of a gas-impermeable material to provide a medical device package; and exposing the medical device package to radiation, thereby sterilizing the inside of the medical device package, the exposure to radiation being performed after at least a reducing gas is enclosed in the medical device made of an ester resin.

According to the above configuration, because the sterilization treatment by exposure to radiation is performed after a reducing gas is enclosed, the degradation of the ester resin forming the medical device is suppressed, and the generation of by-products due to the decomposition of the ester resin can be effectively suppressed. As a result, adverse effects on the quality of the medical device made of an ester resin after sterilization can be avoided.

In the method for sterilizing a medical device made of an ester resin configured as above, the reducing gas may be hydrogen gas; an oxygen scavenger may be further enclosed in the medical device package; a mixed gas comprising the reducing gas and an inert gas may be enclosed in the medical device; and the packaging material may be a film having gas impermeability, and the inert gas may be nitrogen gas. A typical example of the medical device made of an ester resin is a hollow-fiber-type blood treatment device including hollow fibers made of acetyl cellulose.

The above object, other objects, features, and advantages of the present invention will be apparent from the following detailed description of the preferred embodiments taken in conjunction with the accompanying drawings.

### Advantageous Effects of Invention

According to the above configuration, the present invention is advantageous in that in the case where a medical device made of an ester resin, such as a hollow-fiber-type blood treatment device including hollow fibers made of acetyl cellulose, when the medical device is sterilized by exposure to radiation, the degradation of the ester resin and also the generation of by-products, such as acetic acid (carboxylic acid), due to the decomposition of the ester resin can be effectively suppressed.

### DESCRIPTION OF EMBODIMENTS

Hereinafter, preferred embodiments of the present invention will be described. The medical device made of an ester resin in the present invention is made of an ester resin, and examples thereof include various devices used for medical purposes. A typical example thereof is a blood treatment device. However, the medical device made of an ester resin is not limited thereto, and examples thereof also include a bag containing a liquid medicine for in vivo administration. In this embodiment, the present invention will be described in detail taking a blood treatment device as a typical example of the medical device made of an ester resin.

Generally, “blood treatment device” refers to medical instruments used for hemodialysis, hemofiltration, hemodiafiltration, plasma fractionation, plasma separation, etc. A hollow-fiber-type blood treatment device in the present invention refers to an instrument formed of a bundle of fibers made of a synthetic resin, etc., which are called hollow fibers, and a cylindrical container having therein the hollow fiber bundle. It is necessary that such hollow fibers be excellent in terms of characteristics for selectively transmitting a substance in the blood and also in terms of biocompatibility, such as antithrombogenicity.

As a material of hollow fibers that satisfies these conditions, the present invention uses acetyl cellulose, which is an ester resin and also is a cellulose derivative. “Acetyl cellulose” herein is typically triacetylcellulose (TAC), in which the three hydroxyl groups contained in a glucose unit of cellulose are all acetylated (acetic acid is linked to each hydroxyl group by an ester bond), but may also be diacetyl cellulose, etc., in which the ester bonds of some acetyl groups are hydrolyzed back into hydroxyl groups, or an acetyl cellulose composition made of such acetyl cellulose as a main component and also containing other resins, etc., as accessory components.

In addition, the ester resin in the present invention is not limited to acetyl cellulose. Other known resins may also be suitably used as long as they are configured such that a hydroxyl group is contained in the molecule, and the hydroxyl group is linked to an acid, such as a carboxylic acid, by an ester bond (condensation), as long as they are configured to have an ester bond in the molecule. In the present invention, sterilization is performed by exposure to radiation as mentioned later. As long as there is a possibility that the ester bond moiety is cleaved as a result of exposure to radiation, thereby allowing an acid component, such as acetic acid (carboxylic acid), to be released (isolated) as a by-product, any of known ester resins may be the subject of exposure to radiation in the present invention.

Generally, it is preferable that the hollow fibers used for a hollow-fiber-type blood treatment device have an inner diameter within the range of 100 to 300 μm, more preferably within the range of 120 to 250 μm. In addition, it is preferable that the hollow fibers have a thickness within the range of 10 to 50 μm, more preferably within the range of 10 to 30 μm.

The method for modularization as a blood treatment device using the hollow fibers mentioned above is not particularly limited. For example, the following method can be mentioned: generally 7,000 to 12,000 of the hollow fibers are bundled into a hollow fiber bundle and inserted into a cylindrical container of a blood treatment device, a potting agent such as polyurethane is injected into both ends to seal them, then the excess potting agent is cut away together with both ends of the hollow fiber bundle to open the end surfaces of the hollow fibers, and a header is attached thereto.

The specific configurations of various members forming a hollow-fiber-type blood treatment device are not particularly limited, and known members may be suitably used. Incidentally, as members other than the hollow fibers, such as a cylindrical container and a potting agent, those that are unlikely to be degraded by radiation should be used. Examples of materials of a cylindrical container include, but are not particularly limited to, polycarbonate and polypropylene, and examples of materials of a potting agent include, but are not particularly limited to, polyurethane, epoxy resin, and silicone resin.

According to the present invention, a hollow-fiber-type blood treatment device configured as above is hermetically sealed in a packaging material made of a gas-impermeable material, whereby a medical device package is obtained. In this medical device package, at least a reducing gas is enclosed, and an oxygen scavenger is preferably also enclosed.

The packaging material in which a hollow-fiber-type blood treatment device is hermetically sealed should be produced from a gas-impermeable material. The gas-impermeable material is not particularly limited as long as it is a film or sheet having an oxygen permeability of 1 cm³/(m²/24 h/atm) or less and a steam permeability of 5 g/(m²/24 h/atm). However, in the present invention, it is particularly preferable to use a laminate film or sheet having a multilayer structure (multilayer film or multilayer sheet) including an aluminum layer.

The aluminum layer herein may be an aluminum foil or an aluminum deposited layer. In addition, the aluminum layer may be made of 100% aluminum or may also be made of a known aluminum alloy.

Specific examples of a laminate film (or sheet) including an aluminum layer include, but are not particularly limited to, one having a three-layer structure of polyester layer/aluminum layer/polyethylene layer, one having a three-layer structure of polyethylene terephthalate layer/aluminum layer/polyethylene layer, one having a four-layer structure of polyethylene terephthalate layer/polyethylene layer/aluminum layer/polyethylene layer, and one having a four-layer structure of nylon layer/polyethylene layer/aluminum layer/polyethylene layer. Incidentally, the layers of these multilayer structures are described in order from outside to inside.

In these multilayer films (or multilayer sheets), the intermediate layer is an aluminum layer having excellent gas impermeability, and the outer and inner layers are resin layers. Therefore, both functions of gas impermeability and heat sealability can be achieved.

The packaging material configured as above has a bag-like configuration, for example. When a medical device made of an ester resin, such as a hollow-fiber-type blood treatment device, is put into such a bag-like packaging material (bag-like body), and the opening is sealed with a reducing gas being introduced therein, a medical device package can be obtained. Examples of methods for sealing a bag-like body include, but are not particularly limited to, a heat sealing method, an impulse sealing method, a melt sealing method, a frame sealing method, an ultrasonic sealing method, and a high frequency sealing method.

In the present invention, at least a reducing gas is enclosed in the medical device packaged in the packaging material (medical device in the medical device package). When a reducing gas is present in the medical device, even when the medical device package is exposed to radiation, the degradation of acetyl cellulose and the generation of acetic acid can be effectively suppressed. As the reducing gas, in the present invention, it is particularly preferable to use hydrogen gas. However, the reducing gas may also be carbon monoxide, hydrogen sulfide, formaldehyde, etc.

In the present invention, in addition to the reducing gas, an inert gas may also be enclosed in the medical device. In other words, a mixed gas comprising a reducing gas and an inert gas may be enclosed in the medical device. The method for enclosing a reducing gas or a mixed gas in the medical device is not particularly limited, and known enclosure methods, including those using a nozzle, a chamber, etc., may be suitably used.

When the reducing gas is hydrogen gas, the concentration of hydrogen gas can be reduced to decrease flammability or explosiveness, and thus this is particularly preferable. The specific kind of inert gas is not particularly limited, and examples thereof include nitrogen gas, argon gas, helium gas, and carbon dioxide (carbonic acid gas). Among these, it is preferable to use nitrogen gas because of its low cost, etc.

Incidentally, the concentration of the reducing gas in a mixed gas is not particularly limited. When the reducing gas is hydrogen gas, the concentration should at least be 5% by volume or less, preferably about 2% (within the range of 1 to 3%). When the concentration of the reducing gas is within such a range, the flammability of the mixed gas in the medical device, etc., can be effectively reduced.

In the present invention, when at least a reducing gas is enclosed in the medical device in the packaging material, the degradation of the ester resin, such as acetyl cellulose, and the generation of by-products, such as acetic acid (carboxylic acid), can be effectively suppressed. However, it is preferable that an oxygen scavenger is further enclosed in the medical device. In the medical device, a small amount of oxygen may be present. Thus, when an oxygen scavenger is enclosed, the internal oxygen can be selectively removed. Accordingly, the possibility that the internal oxygen molecules are converted into oxygen radicals due to exposure to radiation can be significantly reduced. As a result, the degradation of the ester resin and the generation of by-products caused by oxygen radicals can also be effectively suppressed. In addition, before and after the exposure to radiation, the oxidative degradation of the medical device made of an ester resin due to the presence of oxygen can also be effectively suppressed.

Specific examples of the oxygen scavenger used in the present invention include, but are not particularly limited to, sulfite, hydrogen sulfite, dithionite, hydroquinone, catechol, resorcin, pyrogallol, gallic acid, Rongalite™, ascorbic acid and/or a salt thereof, sorbose, glucose, lignin, dibutylhydroxytoluene, dibutylhydroxyanisole, a ferrous salt, and metal powders such as an iron powder. These oxygen scavengers may be used alone, and it is also possible to use two or more kinds in appropriate combination.

In addition, when the oxygen scavenger is made mainly of a metal powder, a known oxidation catalyst, such as a metal halogen compound, may also be added as necessary. In addition to the oxidation catalyst, the oxygen scavenger may also contain a deodorant, a refresher, and other functional fillers. The form of the oxygen scavenger is not particularly limited either. For example, it may be in the form of a powder, granules, a mass, or a sheet. It is also possible that a substance to serve as an oxygen scavenger is dispersed in a thermoplastic resin and formed into a sheet or a film.

In the present invention, the medical device package configured as above is exposed to radiation to sterilize the inside thereof.

The radiation used for sterilization in the present invention refers to electromagnetic waves or particle rays, such as α-rays, β-rays, γ-rays, electron rays, proton rays, and neutron rays. Among these radiation rays, in terms of sterilization efficiency, handleability, etc., it is preferable to use γ-rays.

The dose of radiation applied to the medical device package should be within a range where sterilization can be achieved, and is generally within the range of 10 to 50 kGy, preferably within the range of 10 to 30 kGy. When the dose of radiation is too low, sufficient sterilization effects may not be obtained. On the other hand, when the dose of radiation is too high, because of the excessive dose, members made of an ester resin (e.g., hollow fibers) or other members of the medical device made of an ester resin may be excessively degraded or decomposed.

The exposure of the medical device package to radiation should be performed at least in a state where the medical device made of an ester resin and the reducing gas are hermetically sealed, and other conditions are not particularly limited. Incidentally, in the case where an oxygen scavenger is further enclosed in the medical device, generally, it is preferable that exposure to radiation be performed when 2 days (48 hours) or more have elapsed after hermetic sealing. This is because, depending on the kind of oxygen scavenger used, the size of the bag-like body, or other conditions, when 48 hours or more are allowed to elapse after hermetically sealing an oxygen scavenger in a bag-like body, the internal oxygen concentration can usually be made negligibly small (usually about 0.1% by volume or less).

However, when the duration between hermetic sealing and exposure to radiation is too long, unwanted bacteria may grow in the medical device package. Therefore, it is preferable that exposure to radiation be performed within 10 days after hermetic sealing at the latest, more preferably within 7 days, and still more preferably within 5 days.

### EXAMPLES

The present invention will be described in further detail by way of examples, comparative examples, and a reference example. However, the prevent invention is not limited thereto. Those skilled in the art can make various changes, amendments, and modifications without deviating from the scope of the present invention. Incidentally, the dialysis membrane eluate test in the following examples, comparative examples, and reference example was performed as follows.

(Dialysis Membrane Eluate Test and Evaluation)

In accordance with the Dialytic Artificial Kidney Device Approval Standards (PAB Notification No. 494), “3. Dialysis Membrane Eluate Test,” an eluate test was performed by the following procedure, and the eluate was evaluated.

First, in a clean environment, a hollow-fiber-type blood treatment device was taken out from a medical device package. The body case was cut using an ultrasonic cutter, and hollow fibers were taken out from the body case. The hollow fibers were cut to a length of 2 cm using a microtome, and a portion weighing 1.5 g was taken to obtain a hollow fiber sample.

Next, the hollow fiber sample was placed in a conical flask containing 150 mL of distilled water, and heated at 70° C. for 1 hour using a constant-temperature water bath. After the completion of heating, followed by cooling, the sample liquid was collected from the conical flask and diluted with distilled water to 150 mL, resulting in a test liquid. Incidentally, to provide a control sample for pH measurement and ultraviolet absorption spectrum measurement, a blank was also prepared by the same procedure using only distilled water.

For the evaluation of the obtained test liquid, the appearance, foaming, pH, and ultraviolet absorption spectrum (UV 220 nm) were evaluated or measured in accordance with standards. Incidentally, for the evaluation of pH, the pH of each test liquid was subtracted from the pH of the blank to calculate ΔpH. The appearance was rated as “◯” (good) when the test liquid was almost transparent and colorless, and no foreign substances were visible to the naked eye; otherwise, a rating of “x” (poor) was given. Foaming was evaluated in accordance with the standards, and a rating of “◯” was given when foams almost disappeared within 3 minutes; otherwise, a rating of “x” was given. The pH of the test liquid was measured using a pH meter (product name: F-24) manufactured by HORIBA, Ltd., while the ultraviolet absorption spectrum of the test liquid was measured using a spectrophotometer (product name: U-3000) manufactured by Hitachi, Ltd.

Further, the elution of heavy metals (elution of copper, zinc, lead, hexavalent chromium, and cadmium) was also evaluated in accordance with standards. However, with respect to the elution of zinc or copper, the test liquid was not pretreated, and measurement was performed using an ICP emission spectrophotometer (product name: OPTIMA8300) manufactured by PerkinElmer, Inc. A rating of “◯” was given in the case where the heavy metal elution volume was not higher than the standard; otherwise, a rating of “x” was given. The calibration curve at this time was prepared by diluting a standard solution for atomic absorption manufactured by Wako Pure Chemical Industries, Ltd., with ultrapure water.

### Example 1

As a packaging material made of a gas-impermeable material, a bag-like body formed of a laminate film (manufactured by Toppan Printing Co., Ltd.) having a three-layer structure including, from the outside, polyethylene terephthalate film/aluminum foil or deposited film/polyethylene film was used. As a hollow-fiber-type blood treatment device, a triacetate hollow fiber dialyzer (Model No. FB-150G) manufactured by Nipro Corporation was used. As an oxygen scavenger, Sansokatto™, which is an iron powder oxygen scavenger manufactured by Iris Fine Products Co., Ltd., was used.

In the ambient atmosphere, one of the above dialyzer and one of the above oxygen scavenger were put into the above bag-like body, and air in the bag-like body was evacuated using a vacuum pump. Subsequently, using a nozzle, a mixed gas was charged into the bag-like body from a mixed gas cylinder containing 2% by volume of hydrogen gas/98% by volume of nitrogen gas (manufactured by Taiyo Nippon Sanso Corporation). This mixed gas charging operation was repeated five times, whereby the bag-like body was sufficiently filled with the mixed gas. Subsequently, the opening of the bag-like body was sealed using a heat sealer to hermetically seal the bag-like body, thereby providing a sample of the medical device package of the present invention. Incidentally, three such samples were produced in total (samples x1, x2, and x3).

After hermetic sealing, the obtained samples were allowed to stand at room temperature for 48 hours or more. Subsequently, the samples were exposed to γ-rays of 15 kGy and thereby sterilized (the sterilization treatment was performed at Koga Isotope, Ltd.). The samples after sterilization were subjected to the dialysis membrane eluate test and evaluation as mentioned above. The results are shown in Table 1.

### Example 2

Three samples of medical device packages in total (samples x1, x2, and x3) were produced in the same manner as in Example 1, except that an oxygen scavenger was not enclosed in the bag-like body.

These samples were also sterilized by exposure to radiation in the same manner as in Example 1, followed by the dialysis membrane eluate test and evaluation. The results are shown in Table 1.

### Comparative Example 1

Three comparative samples of medical device packages in total (samples x1, x2, and x3) were produced in the same manner as in the above examples, except that the evacuation of air from the bag-like body and the charging of a mixed gas were not performed.

These comparative samples were also sterilized by exposure to radiation in the same manner as in Example 1, followed by the dialysis membrane eluate test and evaluation. The results are shown in Table 1.

TABLE 1

| | | | UV 220 | Appear- | | Heavy metal |
|---|---|---|---|---|---|---|
| | | ΔpH | nm | ance | Foaming | elution |
| Example 1 | x1 | 0.92 | 0.023 | ◯ | ◯ | ◯ |
| | x2 | 1.05 | 0.025 | ◯ | ◯ | ◯ |
| | x3 | 1.04 | 0.022 | ◯ | ◯ | ◯ |
| Example 2 | x1 | 0.97 | 0.025 | ◯ | ◯ | ◯ |
| | x2 | 1.03 | 0.026 | ◯ | ◯ | ◯ |
| | x3 | 1.08 | 0.027 | ◯ | ◯ | ◯ |
| Comparative | x1 | 1.16 | 0.027 | ◯ | ◯ | ◯ |
| Example 1 | x2 | 1.45 | 0.031 | ◯ | ◯ | ◯ |
| | x3 | 1.46 | 0.031 | ◯ | ◯ | ◯ |

### Comparative Example 2

Three comparative samples of medical device packages in total (samples x1, x2, and x3) were produced in the same manner as in Example 1, except that POLYNEPHRON™ PES-Sα (Model No. PES-11Sα), which is a polyethersulfone dialyzer manufactured by Nipro Corporation, was used as a hollow-fiber-type blood treatment device, AGELESS™, which is an iron powder oxygen scavenger manufactured by Mitsubishi Gas Chemical Company, Inc., was used as an oxygen scavenger, and 5% by volume of hydrogen gas/95% by volume of nitrogen gas was used as a mixed gas.

These comparative samples were also sterilized by exposure to radiation in the same manner as in Example 1, followed by the dialysis membrane eluate test (for the measurement of ΔpH and ultraviolet absorption spectrum (UV 220 nm)). The results are shown in Table 2.

### Comparative Example 3

Three comparative samples of medical device packages in total (samples x1, x2, and x3) were produced in the same manner as in Comparative Example 2, except that an oxygen scavenger was not enclosed in the bag-like body (that is, in the same manner as in Example 2, except that POLYNEPHRON™ PES-Sα was used as a hollow-fiber-type blood treatment device, and that 5% by volume of hydrogen gas/95% by volume of nitrogen gas was used as a mixed gas).

These comparative samples were also sterilized by exposure to radiation in the same manner as in Example 1, followed by the dialysis membrane eluate test (for the measurement of ΔpH and ultraviolet absorption spectrum (UV 220 nm)). The results are shown in Table 2.

### Comparative Example 4

Three comparative samples of medical device packages in total (samples x1, x2, and x3) were produced in the same manner as in Comparative Example 2, except that the evacuation of air from the bag-like body and the charging of a mixed gas were not performed, and that an ordinary sterilization treatment was performed (that is, in the same manner as in Comparative Example 1, except that POLYNEPHRON™ PES-Sα was used as a hollow-fiber-type blood treatment device).

These comparative samples were also sterilized by exposure to radiation in the same manner as in Example 1, followed by the dialysis membrane eluate test (for the measurement of ΔpH and ultraviolet absorption spectrum (UV 220 nm)). The results are shown in Table 2.

### Reference Example

Three comparative samples in total (samples x1, x2, and x3) were produced in the same manner as in Comparative Example 4. Without performing a sterilization treatment, the samples were subjected to the dialysis membrane eluate test (for the measurement of ΔpH and ultraviolet absorption spectrum (UV 220 nm)). The results are shown in Table 2.

TABLE 2

| | | ΔpH | UV 220 nm |
|---|---|---|---|
| Comparative | x1 | 0.45 | 0.069 |
| Example 2 | x2 | 0.54 | 0.075 |
| | x3 | 0.51 | 0.077 |
| Comparative | x1 | 0.40 | 0.241 |
| Example 3 | x2 | 0.39 | 0.221 |
| | x3 | 0.24 | 0.232 |
| Comparative | x1 | 0.56 | 0.077 |
| Example 4 | x2 | 0.58 | 0.079 |
| | x3 | 0.49 | 0.073 |
| Reference | x1 | 0.14 | 0.113 |
| Example | x2 | 0.03 | 0.161 |
| | x3 | 0.00 | 0.126 |

### Comparison Among Examples, Comparative Examples, and Reference Example

As shown in Table 1, none of the samples of Examples 1 and 2 and Comparative Example 1 was rated as “x” in terms of appearance, foaming, and heavy metal elution. However, the ΔpH was greater in Comparative Example 1 than in Examples 1 and 2 (on average, 1.01 in Example 1, 1.02 in Example 2, and 1.35 in Comparative Example 1), and the ultraviolet absorption spectrum was higher in Comparative Example 1 than in Examples 1 and 2 (on average, 0.023 in Example 1, 0.026 in Example 2, and 0.030 in Comparative Example 1).

That is, in Examples 1 and 2 and Comparative Example 1, components restricted by the approval standards were not eluted from the dialysis membrane. However, it appears that in Comparative Example 1, acetyl cellulose was decomposed due to exposure to radiation to generate acetic acid, resulting in a decrease in pH, and acetic acid was also eluted into the test liquid, resulting in an increase in the ultraviolet absorption spectrum. Meanwhile, it appears that in Examples 1 and 2, because hydrogen gas, which is a reducing gas, was enclosed, the decomposition of acetyl cellulose due to exposure to radiation was suppressed, and thus there was no decrease in pH or an increase in the ultraviolet absorption spectrum caused by the generation of acetic acid.

In addition, no significant difference is seen between the results of Example 1 and Example 2. Accordingly, it appears that the decomposition of acetyl cellulose associated with exposure to radiation was effectively suppressed by the enclosure of the reducing gas. Incidentally, both the ΔpH and the ultraviolet absorption spectrum are lower in Example 1, in which an oxygen scavenger was enclosed, than in Example 2, in which an oxygen scavenger was enclosed. This shows that in the present invention, the decomposition of an ester resin can be effectively suppressed when at least a reducing gas is enclosed, and also that decomposition can be even more suppressed when an oxygen scavenger is enclosed.

Here, Examples 1 and 2 and Comparative Example 1 shown in Table 1 are examples in which a hollow-fiber-type blood treatment device including hollow fibers made of an ester resin is used as mentioned above. Meanwhile, Comparative Examples 2 to 4 and Reference Example shown in Table 2 are examples in which a hollow-fiber-type blood treatment device including no hollow fibers made of an ester resin is used. Of Comparative Examples 2 to 4, Comparative Example 2 corresponds to Example 1, Comparative Example 3 corresponds to Example 2, and Comparative Example 4 corresponds to Comparative Example 1. In addition, Reference Example is a non-sterilized example and thus serves as a standard for evaluating the results of Comparative Examples 2 to 4.

With respect to the ΔpH in Comparative Examples 2 to 4, as shown in Table 2, although the ΔpH values of Comparative Examples 2 to 4 are all greater than in Reference Example, the ΔpH values of Comparative Examples 2 and 4 are comparably greater than the ΔpH of Comparative Example 3 (on average, 0.06 in Reference Example, 0.50 in Comparative Example 2, 0.34 in Comparative Example 3, and 0.54 in Comparative Example 4). Meanwhile, in Examples 1 and 2, the ΔpH values are almost comparable, and the values are smaller than in Comparative Example 1.

In addition, with respect to the ultraviolet absorption spectra of Comparative Examples 2 to 4, as shown in Table 2, unlike ΔpH, the ultraviolet absorption spectra of Comparative Examples 2 and 4 are generally lower than the ultraviolet absorption spectrum of Reference Example, and only the ultraviolet absorption spectrum of Comparative Example 3 is higher than the ultraviolet absorption spectrum of Reference Example (on average, 0.133 in Reference Example, 0.074 in Comparative Example 2, 0.231 in Comparative Example 3, and 0.076 in Comparative Example 4). Meanwhile, in Examples 1 and 2, the ultraviolet absorption spectra are almost comparable, and the values are lower than in Comparative Example 1.

As shown above, the ΔpH values and ultraviolet absorption spectra of Comparative Examples 2 to 4 are different from the ΔpH values and ultraviolet absorption spectra of Examples 1 and 2 and Comparative Example 1. This shows that the application of the present invention to a hollow-fiber-type blood treatment device including no hollow fibers made of an ester resin does not produce effective results. Incidentally, in Examples 1 and 2 and Comparative Examples 2 and 3, although the mixing ratios of the used mixed gases are different, they are all within the above practical range (in the case of hydrogen gas, 5% by volume or less). Therefore, a difference in the mixing ratio of a mixed gas does not substantially affect the difference between the results of these examples and comparative examples.

As shown above, according to the present invention, because sterilization by exposure to radiation is performed after enclosing a reducing gas, the degradation of the ester resin forming the hollow fibers, such as acetyl cellulose, is suppressed, and the generation of by-products, such as acetic acid (carboxylic acid), due to the decomposition of the ester resin can be effectively suppressed. As a result, the drug product placed into the medical device, the drug product that is about to be administered, or the patient's blood can be prevented from the unintended effect of acetic acid (carboxylic acid), etc., released by the decomposition. In addition, it is also possible to avoid adverse effects on the quality of the medical device, such as a hollow-fiber-type blood treatment device, after sterilization.

That is, in a resin having an ester bond involved in the main chain, the problem in which an acid resulting from decomposition serves as a catalyst to hydrolyze an ester, whereby the ester bond is cleaved, resulting in a decrease in the molecular weight and a loss of strength, is unlikely to occur. Accordingly, the possibility of defects, such as trouble due to stress applied upon use, can be reduced. In addition, in a resin having an ester bond in the side chain, changes in the surface charge or stereochemical structure due to decomposition, and the resultant unintended interaction with the patient's blood or the drug product, are prevented.

Various modifications and other embodiments of the present invention will be apparent to those skilled in the art from the above description. Thus, the above description should be considered as examples only, and they are intended to teach those skilled in the art the best mode for carrying out the present invention. Without deviating from the spirit of the present invention, the details of the structure and/or function may be substantially changed.

### INDUSTRIAL APPLICABILITY

The present invention is widely and suitably applicable to the field of sterilization and production of a medical device made of an ester resin including a member made of an ester resin that may be degraded or decomposed due to exposure to radiation, such as a hollow-fiber-type blood treatment device including hollow fibers made of acetyl cellulose.

1. A method for sterilizing a medical device made of an ester resin, comprising:
hermetically sealing a medical device made of an ester resin in a packaging material made of a gas-impermeable material to give a medical device package; and
exposing the medical device package to radiation, thereby sterilizing the inside of the medical device package,
the exposure to radiation being performed after at least a reducing gas is enclosed in the medical device made of an ester resin.

2. The method for sterilizing a medical device made of an ester resin according to claim 1, wherein the reducing gas is hydrogen gas.

3. The method for sterilizing a medical device made of an ester resin according to claim 1, wherein an oxygen scavenger is further enclosed in the medical device package.

4. The method for sterilizing a medical device made of an ester resin according to claim 1, wherein a mixed gas of the reducing gas and an inert gas is enclosed in the medical device package.

5. The method for sterilizing a medical device made of an ester resin according to claim 4, wherein
the packaging material is a film having gas impermeability, and
the inert gas is nitrogen gas.

6. The method for sterilizing a medical device made of an ester resin according to claim 1, wherein the medical device made of an ester resin is a hollow-fiber-type blood treatment device including hollow fibers made of acetyl cellulose.
